(19) United States
(12) Patent Application Publication
(10) Pub. No.: US 2021/0369720 A1
(43) Pub. Date: Dec. 2, 2021

(54) SYNERGISTIC COMPOSITION HAVING NEUROPROTECTIVE PROPERTIES AND METHODS OF USE THEREOF

(71) Applicants: Rutgers, The State University of New Jersey, New Brunswick, NJ (US); The Trustees of Princeton University, Princeton, NJ (US)

(72) Inventors: Mary M. Mouradian, Princeton, NJ (US); Jeffry B. Stock, Princeton, NJ (US)

(73) Assignees: Rutgers, The State University of New Jersey, New Brunswick, NJ (US); The Trustees of Princeton University, Princeton, NJ (US)

(21) Appl. No.: 17/290,870

(22) PCT Filed: Nov. 1, 2019
(86) PCT No.: PCT/US2019/059435

§ 371 Date: May 3, 2021

Related U.S. Application Data
(60) Provisional application No. 62/755,074, filed on Nov. 2, 2018.

(51) Int. Cl. A61K 31/522; A61K 31/4045; A61K 45/06; A61P 25/28

(52) U.S. Cl. CPC A61K 31/522; A61P 25/28; A61K 45/06; A61K 31/4045

(57) ABSTRACT

A neuroprotective compositions containing caffeine or a caffeine analogue and a long chain fatty acyl tryptamide with an aliphatic chain having 16 to 22 carbons linked to a tryptamine, wherein the composition contains from at least 1.5 mg to 600 mg of caffeine per serving or unit dosage of the composition; from at least 0.5 mg to 300 mg of the long chain fatty acyl tryptamide per serving or unit dosage of the composition; and the ratio of long chain fatty acyl tryptamide to caffeine is from 1:1200 to 200:1. Methods are also disclosed for treating or preventing cognitive and movement deficits of a disease, condition or disorder or neurological deterioration that use the disclosed neuroprotective compositions.

### CROSS-REFERENCE TO RELATED APPLICATION

This application claims the benefit of U.S. Provisional Application No. 62/755,074 filed Nov. 2, 2018, the disclosure of which is incorporated herein by reference in its entirety.

### STATEMENT REGARDING FEDERALLY SPONSORED RESEARCH

The invention described herein was sponsored in whole or in part by a grant from the National Institutes of Health (NIH), namely NIH grant_AT006868.

### FIELD OF THE INVENTION

This invention relates to compositions that have neuroprotective properties, more specifically synergistic compositions containing a long chain fatty acyl tryptamide and caffeine.

### BACKGROUND OF THE INVENTION

Neurodegenerative diseases include, among other things, Parkinson's disease, Parkinson's disease dementia, Lewy Body Dementia, and Alzheimer's disease. Most neurodegenerative diseases begin in the middle to later years of life and lead to progressive degeneration of the brain, ultimately resulting in premature death.

Parkinson's disease (PD) and dementia with Lewy bodies (DLB) are two of the most prevalent neurodegenerative diseases. PD can be inherited due to various genetic mutations or it can be sporadic with no known identifiable cause. Environmental factors such as well water drinking have been implicated as a contributing factor. The disorder generally begins with tremors, slow movements, stiff joints, and can progress to shuffling gait, and eventually inability to walk, and incapacitation. In the advanced stages, the disease is frequently accompanied by dementia. Dementia with Lewy bodies is a type of progressive dementia that leads to progressive cognitive decline, fluctuations in alertness and attention, visual hallucinations, and parkinsonian motor symptoms, such as slowness of movement, difficulty walking, or rigidity. These symptoms of PD and DLB are caused by loss of brain neurons that contain hallmark inclusions known as Lewy bodies.

Both Parkinson's disease and dementia with Lewy bodies are characterized by the presence of Lewy bodies that are seen under the microscope in postmortem brains. Lewy bodies contain clumps of the protein α-synuclein. In addition, α-synuclein pathology is found in another neurodegenerative disorder known as Multiple System Atrophy (MSA), and these three disorders collectively are referred to as “α-synucleinopathies.” Alzheimer's disease affected brains can also have α-synuclein pathology on postmortem examination in addition to amyloid plaques and tau containing neurofibrillary tangles. Accordingly, α-synuclein and tau proteins are of great interest to researchers because of their role in several neurodegenerative diseases. Excessive phosphorylation of α-synuclein in the Lewy bodies and Lewy neurites has been found to be a characteristic neuropathological feature of both Parkinson's disease (PD) and dementia with Lewy bodies (DLB) Similarly, excessive phosphorylation of tau protein is a common characteristic of Alzheimer's disease, progressive supranuclear palsy, and Chronic Traumatic Encephalopathy (CTE). Thus, mechanisms to decrease α-synuclein and tau protein phosphorylation may have therapeutic benefit.

Levodopa, dopamine agonists, monoamine oxidase (MAO) inhibitors, Catechol-O-Methyltransferase (COMT) inhibitors, and anticholinergics are frequently administered to modify neural transmissions and thereby suppress the symptoms of PD, however, there is no known therapy which halts or slows down the underlying progression of the neurodegenerative process. Similarly, there are no known cures that slow the progression of DLB or MSA, or a number of other neurodegenerative diseases.

Some epidemiological studies have suggested an inverse association between coffee consumption and the risk of PD and Alzheimer's disease, among other things. Various mechanisms for the purported benefits have been suggested, but none have been explored fully enough for these suggestions to be definitive. Caffeine is generally believed to be the neuroprotective agent in coffee. Prior studies about the protective potential of coffee in PD have focused largely on caffeine, because epidemiological data are consistent with caffeine as a major source of neuroprotective activity. However, among patients with early PD, the amount of caffeine consumed does not impact the rate of progression of the disease, and decaffeinated coffee has been found to be protective in Drosophila models of PD, raising some question about the protective effect of only caffeine among the numerous other compounds in coffee. Tryptamides, which can also be found in coffee, have proven efficacy in various models of neurodegeneration, including for example, Parkinson and Alzheimer diseases. They have been previously discovered to modulate protein phosphatase 2A (PP2A), which is able to dephosphorylate α-synuclein, to enhance the health, and various cognitive functions, of the brain.

However, there remains a need for compositions that can provide neuroprotection to prevent, slow down, or treat neurodegenerative diseases and conditions. Additionally, there is a need for compositions that can provide a level of neuroprotection which surpasses that which can be obtained with caffeine alone or tryptamides alone.

### SUMMARY OF THE INVENTION

The invention described herein involves compositions having neuroprotective properties and methods of use of such compositions.

According to one embodiment of the present invention, a neuroprotective composition comprising caffeine and a long chain fatty acyl tryptamide comprising an aliphatic chain having 16 to 22 carbons linked to a tryptamine, wherein the composition contains at least 1.5 mg to 600 mg of caffeine per serving or unit dosage of the composition; wherein the composition contains from at least 0.5 mg to 300 mg of the long chain fatty acyl tryptamide per serving or unit dosage of the composition; and wherein the ratio of long chain fatty acyl tryptamide to caffeine is from 1:1200 to 200:1, is provided. According to a different embodiment, the composition contains from at least 5 mg to 20 mg of caffeine per serving or unit dosage of the composition, and from at least 0.5 to 10 mg of the long chain fatty acyl tryptamide per serving or unit dosage of the composition. In another embodiment, the ratio of long chain fatty acyl tryptamide to caffeine is from 1:3 to 1:150.

According to another embodiment, the long chain fatty acyl tryptamide in the composition of the invention is saturated. In another embodiment of the invention, the tryptamine that is linked to the aliphatic chain on the long chain fatty acyl tryptamide is a 5-hydroxytryptamine. According to another embodiment, the long chain fatty acyl tryptamide in the composition of the invention is eicosanoyl-5-hydroxytryptamide.

According to another embodiment, the composition consists essentially of the caffeine, the long chain fatty acyl tryptamide, and at least one of, a pharmaceutically acceptable carrier, excipient, electrolyte, legal stimulant, vitamin, mineral, or health supplement. According to another embodiment, the pharmaceutically acceptable carrier is selected from the group consisting of liposomes, polymeric micelles, microspheres, nano structures, nanofibers, and dendrimers. According to another embodiment, the pharmaceutically acceptable excipient is selected from the group consisting of microcrystalline cellulose, dicalcium phosphate, stearic acid, magnesium stearate, croscarmellose sodium, silicon dioxide, enteric coating, natural flavors, gelatin, titanium dioxide, white rice flour, salt, acetic acid, disodium EDTA, rice bran oil, vegetable wax, gelatin, glycerin, water, colors, cellulose, pharmaceutical glaze, starch, maltodextrin, vegetable cellulose, sunflower lecithin, safflower oil, glycerin, sunflower lecithin, sorbitol, and modified food starch. According to another embodiment, the active ingredients of the composition consist of the long chain fatty acyl tryptamide and the caffeine. According to another embodiment, the long chain fatty acyl tryptamide and the caffeine are in the form of nanoparticles or microparticles. According to another embodiment, the nanoparticles or microparticles have a diameter of from at least 10 nm to no more than 500 nm. According to another embodiment, the long chain fatty acyl tryptamide and the caffeine exhibit a synergistic effect in at least one of preventing, reducing or controlling the formation of α-synuclein aggregates.

According to another embodiment of the invention, a method of treating or prophylactically treating patients at risk of suffering from movement or cognitive effects of a disease, condition or disorder selected from the group consisting of Alzheimer's disease, Mild Cognitive Impairment, Parkinson's disease, Parkinson's disease dementia, Lewy Body Dementia, Progressive Supranuclear Palsy, Multisystem Atrophy, Corticobasal Degeneration, Frontotemporal Dementia, Huntington's disease, Amyotrophic Lateral Sclerosis, Spinocerebellar Ataxia, Friedrich's Ataxia, bipolar disorder, cerebrovascular disorder, encephalopathy, traumatic brain injury, Chronic Traumatic Encephalopathy, multiple sclerosis, and other demyelinating and inflammatory disorders of the nervous system, comprising administering the composition of the claimed invention at a dosage of at least 0.5 mg of the long chain fatty acyl tryptamide, and at least 1.5 mg of the caffeine, is provided. According to another embodiment, the long chain fatty acyl tryptamide used in the method of the invention is eicosanoyl-5-hydroxytryptamide. According to another embodiment, the ratio of long chain fatty acyl tryptamide to caffeine used in the method is from 1:1200 to 200:1. According to another embodiment, the neuroprotective composition of the invention is administered in a form selected from the group consisting of a beverage, foodstuff, chewing gum, candy, chocolate bar, pharmaceutical composition, nutraceutical or nutritional supplement. According to another embodiment, the beverage used in the method is selected from the group consisting of water, a fruit drink, coffee, tea, energy drink, nutritional drink or sport drink. According to another embodiment, the pharmaceutical composition is administered in the form of a powder, tablet, capsule, dissolving strips, lozenge, syrup, suspension, emulsion, tincture, elixir or effervescent formulation.

According to yet another embodiment of the invention, a method of prophylactically treating patients at risk of developing neurological disorder or improving a neurological disorder in a subject in need thereof including the steps of administering a composition containing a long chain fatty acyl tryptamide in amounts of at least 0.5 mg and the caffeine in amounts of at least 1.5 mg, wherein the neurological disorder is selected from the group consisting of, decline in memory, mild cognitive impairment, decline in executive function, dementia, reduced alertness, slow movements, Parkinsonian signs, tremor, poor coordination of movements, anosmia, REM sleep behavior disorder, or any genetic locus identified as a risk factor for neurodegenerative disease, is provided.

According to a further embodiment of the invention, a method of reducing at least one of α-synuclein aggregation or tau protein aggregation in the nervous system tissue of a subject in need thereof, comprising administering the composition according to any one of the methods of the other embodiments of the invention, is provided.

According to still a further embodiment of the invention, a method of reducing at least one of phosphorylated α-synuclein aggregate levels or phosphorylated tau protein aggregate levels in central and peripheral tissues of a subject in need thereof is described wherein the method comprises administering the composition of the present invention. According to another embodiment, a method according to any one of other embodiments of the invention, wherein the tissue has a pathology selected from the group consisting of Lewy bodies, Lewy neurites, neurofibrillary tangles, amyloid plaques, or other pathologic protein aggregates or inclusions, is provided.

According to another embodiment of the invention, a method of reducing the levels of inflammatory markers in a subject in need thereof, including the step of administering the composition of the present invention in effective amounts to reduce the inflammatory markers, is provided. According to another embodiment, the inflammatory markers are representative of at least one of microgliosis or astrocytosis, is provided.

According to a different embodiment of the invention, a method of increasing the levels of dopamine in the brain of a subject in need thereof, including the step of administering the composition of the invention in effective amounts to increase the dopamine, is provided.

According to a yet another embodiment of the invention, a method of protecting and increasing tyrosine hydrolase (TH) positive dopaminergic neurons in a subject in need thereof, including the step of administering the composition of the invention in effective amounts to increase TH positive dopaminergic neurons, is provided.

According to a further embodiment of the invention, a method of increasing the levels of methylated protein phosphatase 2A (PP2A) in a subject in need thereof, including the step of administering the composition of the invention in effective amounts to increase the methylated PP2A levels, is provided.

According to a still further embodiment of the invention, a method of decreasing the levels of demethylated protein phosphatase 2A (PP2A) in a subject in need thereof, including the step of administering the composition of the invention in effective amounts to decrease the demethylated PP2A levels, is provided.

### BRIEF DESCRIPTION OF THE FIGURES

FIG. 1 depicts the results of four behavioral performance tests: Rotarod (A), Wire Hang (B), Nesting behavior (C) and Morris Water Maze (D) conducted on six month old wild type (WT) and α-synuclein transgenic (Syn^{Tg}) mice, demonstrating that EHT and Caffeine co-treatment, given at doses that are individually ineffective therapeutically, prevents the behavioral deficits of Syn^{Tg} mice.

FIG. 2 depicts quantification of immunohistochemical staining of phosphorylated-α-synuclein (p-α-Syn) in the cortex (A) and hippocampus (B); (C) quantification of p-α-Syn levels in cortical brain tissue lysates from five groups and five animals per group determined by Western blotting; and quantification of: (D) immunofluorescence staining of MAP2 in the cortex; (E) immunohistochemical staining of c-fos in the hippocampus; (F) immunohistochemical staining of Iba-1 in the striatum; and (G) immunohistochemical staining of GFAP in the cortex, demonstrating that EHT and caffeine co-treatment reduces p-α-Syn burden and protects against the neuronal damage and neuroinflammation in Syn^{Tg} mice.

FIG. 3 depicts the results of four behavioral performance tests (Rotarod (A), Wire Hang (B), Nesting behavior (C) and Morris Water Maze (D)) conducted on phosphate buffered saline (PBS) and α-Syn preformed fibrils (PFF) inoculated mice at 6 months post-inoculation, and 8 months of age, demonstrating that EHT and CAF co-treatment improves the behavioral performance of α-Syn PFF inoculated WT mice.

FIG. 4 depicts in α-Syn PFF inoculated WT mice quantification of immunohistochemical staining of p-α-Syn in the ipsilateral striatum (A) and contralateral striatum (B); quantification of (C) immunohistochemical staining of p-α-Syn in the ipsilateral substantia nigra pars compacta (SNc); quantification of immunohistochemical staining of Iba-1 in the ipsilateral (D) and contralateral striatum (E), demonstrating that EHT and CAF co-treatment prevents the formation of p-α-Syn positive aggregates and mitigates neuroinflammation.

FIG. 5 depicts in α-Syn PFF inoculated WT mice quantification of (A) ipsilateral and (B) contralateral striatal TH staining; (C) dopamine content in the ipsilateral striatum analyzed by HPLC-MS; and (D) nigral TH positive neuron count, demonstrating that EHT and CAF co-treatment protects nigrostriatal neurons in the α-Syn PFF inoculation model.

FIG. 6 depicts EHT and CAF exert their synergistic neuroprotective effects through regulating PP2A methylation and activity in Syn^{Tg} mice. (A) to (C) are densitometric analyses of Western blots for the indicated proteins with striatal tissue lysates from Syn^{Tg} mouse brains, with bar graphs showing methylated PP2A (A) and demethylated PP2A (B) levels that are normalized to total PP2A, and the ratio of methylated PP2A over demethylated PP2A (C); (D) to (F) Densitometric analyses of Western blots of ipsilateral striatal tissue lysates from α-Syn PFF inoculated mice probed for the indicated proteins, with bar graphs showing methylated PP2A (D) and demethylated PP2A (E) levels that are normalized to total PP2A; and the ratio of methylated PP2A over demethylated PP2A (F), demonstrating that EHT and CAF exert their synergistic neuroprotective effects through regulating PP2A methylation and activity.

FIG. 7 depicts the results of Western blot analysis of SH-SY5Y cell lysates for the indicated proteins, with (A) showing bar graphs of p-Syn (relative to β-actin), (B) methyl-PP2A, (C) de-methyl-PP2A, (D) ratio of methyl PP2A over de-methyl PP2A, demonstrating that the combination of EHT and CAF has synergistic effects in up-regulating PP2A methylation. Panel (E) shows propidium iodide (PI) and DAPI staining of SH-SY5Y cells incubated with PBS or mouse α-Syn PFF and treated with CAF and/or EHT for seven days, demonstrating that the combination of EHT and CAF has synergistic effects in attenuating cytotoxicity induced by α-Syn PFF. All bar graphs represent means±SEM. *p<0.05; **p<0.01; ***p<0.001. EHT=eicosanoyl-5-hydroxytryptamide; CAF=caffeine.

### DETAILED DESCRIPTION OF THE INVENTION

Unless otherwise defined, all technical and scientific terms used herein have the same meaning as commonly understood by one of ordinary skill in the art to which this invention pertains. In the case of conflict, the present document, including definitions, will be controlling.

The present invention provides a synergistic neuroprotective composition that reduces, prevents or ameliorates neurodegeneration in subjects suffering from neurodegenerative diseases, such as Alzheimer's disease, Mild Cognitive Impairment, Parkinson's disease, Parkinson's disease dementia, Lewy Body Dementia, Progressive Supranuclear Palsy, Multisystem Atrophy, Corticobasal Degeneration, Frontotemporal Dementia, Huntington's disease, Amyotrophic Lateral Sclerosis, Spinocerebellar Ataxia, Friedrich's Ataxia, bipolar disorder, cerebrovascular disorder, encephalopathy, traumatic brain injury, Chronic Traumatic Encephalopathy, multiple sclerosis, and other demyelinating and inflammatory disorders of the nervous system, among others, or in subjects seeking to prevent, reduce or delay the onset of neurodegeneration. This invention also concerns methods of using the synergistic compositions to treat or prevent various conditions and diseases and provides novel pharmacological interventions that can lead to reduced phosphorylation of both α-synuclein and tau proteins, which can retard nucleation and propagation of pathology and slow down the progression of such neurodegenerative diseases.

Coffee is by far the most widely and highly consumed herbal extract. Numerous epidemiological studies indicate that coffee consumption affords reduced risk of Parkinson's and Alzheimer's diseases. This association has been attributed to caffeine, but the finding that caffeine may be neuroprotective in no way excludes the possibility that other components in coffee may play a role with caffeine. Studies indicate that the actions of caffeine stem from its antagonism of adenosine A2A receptor signaling, but downstream neuroprotective mechanisms remain to be established. While seeking to elucidate the molecular mechanisms of neuroprotection mediated by caffeine and other components of coffee, a lipid-like component of coffee, eicosanoyl-5-hydroxytryptamide (EHT), was isolated and found to have a protective effect in mouse models of PD. It was unexpectedly discovered that caffeine works synergistically with long chain fatty acyl tryptamides, such as, for example, EHT, to provide neuroprotection that is superior to that which can be obtained by caffeine or EHT alone. Biochemical and neuropathological analyses demonstrated that consumption of the composition of the invention leads to at least one of, decreased α-synuclein phosphorylation and aggregation, a robust anti-inflammatory effect, and protection of neurons against pathologic α-synuclein fibrils, among other things.

The neuroprotective composition of the invention contains caffeine and a long chain fatty acyl tryptamide. Caffeine is a xanthine alkaloid found naturally in coffee beans, tea, kola nuts, Yerba mate, guarana berries, and the like. Chemically, caffeine is 1,3,7-trimethylxanthine, and the chemical formula is C₈H₁₀N₄O₂. It is also known as trimethylxanthine, thein, mateine, guaranine, and methyltheobromine. Caffeine is an adenosine A_{2A} receptor antagonist. When isolated in pure form, caffeine is a white crystalline powder that tastes very bitter. In one embodiment of the invention, the caffeine may be extracted from the fruit of a species of the plant genus Coffea. The caffeine may be prepared by extracting coffee beans, the fruit of the coffee tree, either green, roasted or otherwise treated, of C. arabica, C. robusta, C. liberica, C. arabusta, or other species. The extraction procedure concentrates or isolates the caffeine. Alternatively, the caffeine may be purchased from a commercial source (e.g. Sigma-Aldrich).

In a different embodiment of the invention the caffeine is present in the composition of the invention in the form of a caffeine salt. Suitable salts include, for example, caffeine citrate, caffeine sodium benzoate, caffeine sodium salicylate, and the like.

In one embodiment of the invention, the caffeine or salt thereof is present in the neuroprotective composition in quantities of from at least 1.5 mg to no more than 600 mg of caffeine per serving or unit dosage of the composition. In a preferred embodiment of the invention, the neuroprotective composition contains at least 5 mg of caffeine per serving or unit dosage of the composition, more preferably from 5 mg to 560 mg of caffeine per serving or unit dosage of the composition, and even more preferably from about 5 mg to about 20 mg of caffeine per serving or unit dosage of the composition, and yet more preferably from about 5 mg to about 15 mg of caffeine per serving or unit dosage of the composition. In a more preferred embodiment of the invention, the composition contains at least 10 mg of caffeine per serving or unit dosage of the composition, even more preferably from 10 mg to 500 mg of caffeine per serving or unit dosage of the composition.

In a different embodiment of the invention, the caffeine or salt thereof is present in the neuroprotective composition in quantities of at least 15 mg of caffeine per serving or unit dosage of the composition. In another embodiment of the invention the composition contains at least 25 mg of caffeine per serving or unit dosage of the composition. In still another embodiment of the invention the composition contains at least 35 mg of caffeine per serving or unit dosage of the composition. In yet another embodiment of the invention, the composition contains at least 100 mg of caffeine per serving or unit dosage of the composition. In a further embodiment of the invention, the composition contains at least 200 mg of caffeine per serving or unit dosage of the composition. In a different embodiment of the invention, the composition contains at least 300 mg of caffeine per serving or unit dosage of the composition. In another embodiment of the invention, the composition contains at least 400 mg of caffeine per serving or unit dosage of the composition. In yet another embodiment of the invention, the composition contains at least 500 mg of caffeine per serving or unit dosage of the composition. In yet another embodiment of the invention the composition contains no more than 500 mg of caffeine per serving or unit dosage of the composition. In further embodiment of the invention. the composition contains no more than 560 mg of caffeine per serving or unit dosage of the composition. In another embodiment of the invention, the composition contains no more than 600 mg of caffeine per serving or unit dosage of the composition. In a preferred embodiment of the invention, the amount of caffeine in the composition of the invention is less than the amount of caffeine that occurs naturally, for example in a coffee bean or tea leaf and the like, or in a drink prepared from a coffee bean, tea leaf, and the like.

Alternatively, the long chain fatty acyl tryptamide of the invention may be used in combination with an adenosine A2A receptor antagonist. In one embodiment of the invention, the neuroprotective composition of the invention contains the long chain fatty acyl tryptamide and an adenosine A2A receptor antagonist such as, for example, istradefylline, substituted 5-amino-pyrazolo-[4,3-e]-1,2,4-triazolo [1,5-c]pyrimidine adenosine, as disclosed in U.S. Pat. No. 6,630,475 incorporated herein in its entirety.

Furthermore, the long chain fatty acyl tryptamide of the invention may be used in combination with a different methylxanthine and salts thereof, such as for example, theophylline, theobromine, aminophylline, dyphylline, and combinations thereof.

In yet another embodiment of the invention, the neuroprotective composition contains a long chain fatty acyl tryptamide, and an analogue of caffeine, such as for example, 7-allyl-1,3-dimethylxanthine, 3,7-dimethyl-1-n-propylxanthine, 1,3-dimethyl-7-propargylxanthine.

The caffeine or caffeine analogues and long chain fatty acyl tryptamide may be present in the composition in any suitable form, which acceptable forms are known to those skilled in the art. According to one embodiment, the long chain fatty acyl tryptamide and the caffeine are in the form of nanoparticles or microparticles. According to another embodiment, the nanoparticles or microparticles have a diameter of from at least 10 nm to no more than 500 nm.

The long chain fatty acyl tryptamide has a fatty acyl group, having a long aliphatic hydrocarbon chain that is linked to the tryptamine entity by an amide linkage, forming the tryptamide. By “long chain” is meant that the aliphatic chain on the fatty acyl group has at least 16 carbons on the chain. In a preferred embodiment, the aliphatic chain has from 16 to 22 carbons. The fatty acyl aliphatic chain may be saturated or unsaturated. In a preferred embodiment of the invention, it is saturated. The carboxyl of the fatty acyl group, which derives from a fatty acid, connects to the target amine of a tryptamine to form a tryptamide. As used herein, tryptamides refer to compounds that are encompassed within the formula:

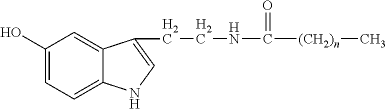

wherein “n” is 14-20, and one or more of the CH₂ groups in the (CH₂)ₙ group can optionally be replaced with CH to provide one or more double bonds.

Tryptamine is a monoamine alkaloid. It contains an indole ring structure and is structurally similar to the amino acid tryptophan, from which the name derives. Tryptamine is found in trace amounts in the brains of mammals and is hypothesized to play a role as a neuromodulator or neurotransmitter. The chemical formula for tryptamine is C₁₀H₁₂N₂. In a preferred embodiment of the invention, the tryptamine is 5-hydroxytryptamine.

As noted above, the tryptamine links, via an amide chain linkage, with the aliphatic chain of the long chain fatty acyl group, forming a tryptamide. Tryptamides modulate protein phosphatase 2A (PP2A) to enhance the health and various cognitive functions of the brain. Trace amounts of tryptamides can be found in coffee and cocoa products, for example in the form of eicosanoyl, docosanyl, and tetracosanoyl 5-hydroxytryptamides. In another embodiment of the invention, the tryptamide is 5-hydroxytryptamide. In a preferred embodiment of the invention, the long chain fatty acyl tryptamide is eicosanoyl-5-hydroxytryptamide, a long chain fatty acyl hydroxytryptamide that has 20 carbons on the acyl chain, and which has the following formula:

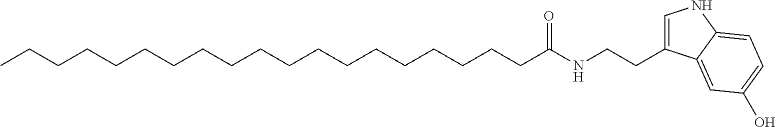 [SMILES: CCCCCCCCCCCCCCCCCCCC(=O)NCCc1c[nH]c2ccc(O)cc12]

The long chain fatty acyl tryptamide of the invention may be extracted from an organic material, such as, for example, coffee beans. Alternatively, it may also be prepared synthetically.

It was unexpectedly discovered that when used in combination with caffeine, tryptamides and caffeine behave synergistically, and provide improved neuroprotection that exceeds what can be provided by a tryptamide alone or caffeine alone. According to one embodiment of the invention, the long chain fatty acyl tryptamide and the caffeine exhibit a synergistic effect in at least one of preventing, reducing or controlling the formation of α-synuclein aggregates (including high-molecular weight aggregates). The synergistic composition may also reduce levels of α-synuclein phosphorylation, or inflammation, or improve or restore hippocampal neuronal activity, or a combination thereof. Thus, the composition of the invention may be useful for reducing, preventing or at least partially reversing the neurodegeneration associated with a variety of conditions such as Alzheimer's disease, Mild Cognitive Impairment, Parkinson's disease, Parkinson's disease dementia, Lewy Body Dementia, Progressive Supranuclear Palsy, Multiple System Atrophy, Corticobasal Degeneration, Frontotemporal Dementia, Huntington's disease, Amyotrophic Lateral Sclerosis, Spinocerebellar Ataxia, Friedrich's Ataxia, bipolar disorder, cerebrovascular disorder, traumatic brain injury, Chronic Traumatic Encephalopathy, encephalopathy, multiple sclerosis, other demyelinating and inflammatory disorders of the nervous system, and the like.

The neuroprotective qualities of the tryptamide-caffeine composition may be enhanced when the tryptamide in the tryptamide-caffeine co-treatment is fortified, meaning that it is present in the composition in quantities that surpass that in which tryptamides are found in natural materials, such as coffee beans and the like, and drinks made from coffee beans and the like. The neuroprotective composition of the invention contains at least 0.05 mg of the long chain fatty acyl tryptamide per serving or unit dosage of the composition. In a preferred embodiment, the composition contains more than 0.1 mg, per serving or unit dosage of the composition, of the long chain fatty acyl tryptamide. In a more preferred embodiment, the composition contains at least 0.5 mg, per serving or unit dosage of the composition, of the long chain fatty acyl tryptamide. In another preferred embodiment, the composition contains from about 0.5 mg to about 10 mg, per serving or unit dosage of the composition, of the long chain fatty acyl tryptamide. In a different embodiment of the invention, the composition contains at least 0.75 mg, per serving or unit dosage of the composition, of the long chain fatty acyl tryptamide. In a further embodiment of the invention, the composition contains at least 1 mg, per serving or unit dosage of the composition, of the long chain fatty acyl tryptamide. In a still further embodiment of the invention, the composition contains at least 5 mg, per serving or unit dosage of the composition, of the long chain fatty acyl tryptamide. In yet another embodiment of the invention, the composition contains at least 10 mg, per serving or unit dosage of the composition, of the long chain fatty acyl tryptamide. In a different embodiment of the invention, the composition contains at least 15 mg, per serving or unit dosage of the composition, of the long chain fatty acyl tryptamide. In another embodiment of the invention, the composition contains at least 20 mg, per serving or unit dosage of the composition, of the long chain fatty acyl tryptamide. In yet another embodiment of the invention, the composition contains at least 40 mg, per serving or unit dosage of the composition, of the long chain fatty acyl tryptamide. In still another embodiment of the invention, the composition contains at least 70 mg, per serving or unit dosage of the composition, of the long chain fatty acyl tryptamide. In a further embodiment of the invention, the composition contains at least 100 mg, per serving or unit dosage of the composition, of the long chain fatty acyl tryptamide. In yet a further embodiment of the invention, the composition contains at least 125 mg, per serving or unit dosage of the composition, of the long chain fatty acyl tryptamide. In another embodiment of the invention, the composition contains at least 150 mg, per serving or unit dosage of the composition, of the long chain fatty acyl tryptamide. In a different embodiment of the invention, the composition contains at least 200 mg, per serving or unit dosage of the composition, of the long chain fatty acyl tryptamide. In another, different embodiment of the invention, the composition contains at least 250 mg, per serving or unit dosage of the composition, of the long chain fatty acyl tryptamide. In a different embodiment of the invention, the composition contains at least 300 mg, per serving or unit dosage of the composition, of the long chain fatty acyl tryptamide.

At least one aspect of the present invention is directed to methods of administering neuroprotective compositions to patients. The method includes the steps of identifying patients who are at risk of developing a neurological disorder, whether for purposes of treatment or prophylactic treatment, and then treating such subjects with compositions containing specific amounts of caffeine and a long chain fatty acyl tryptamide which includes an aliphatic chain having 16 to 22 carbons linked to a tryptamine. In one embodiment, the composition contains from at least 1.5 mg to 600 mg of caffeine per serving or unit dosage of the composition and from at least 0.05 mg to 300 mg of the long chain fatty acyl tryptamide per serving or unit dosage of the composition. In a preferred embodiment, the composition contains from 1.5 mg to 600 mg of caffeine per serving or unit dosage of the composition. In a different preferred embodiment, the composition contains from 5 mg to 560 mg of caffeine per serving or unit dosage of the composition. In another preferred embodiment, the composition contains from 10 mg to 500 mg of caffeine per serving or unit dosage of the composition. In a preferred embodiment, the composition contains from 0.5 mg to 300 mg of the long chain fatty acyl tryptamide per serving or unit dosage of the composition. In a different preferred embodiment, the composition contains at least 0.5 mg of the long chain fatty acyl tryptamide per serving or unit dosage of the composition.

In a preferred embodiment of the invention, the ratio of long chain fatty acyl tryptamide to caffeine ranges from 1:3 to 1:150. In another preferred embodiment of the invention, the ratio of long chain fatty acyl tryptamide to caffeine ranges from 1:5 to 1:40. In a different preferred embodiment of the invention, the ratio of long chain fatty acyl tryptamide to caffeine ranges from 1:6.5 to 1:30. In yet another preferred embodiment of the invention, the ratio of long chain fatty acyl tryptamide to caffeine ranges from 1:8 to 1:25. In a further preferred embodiment of the invention, the ratio of long chain fatty acyl tryptamide to caffeine ranges from 1:10 to 1:20.

In another embodiment, the ratio of long chain fatty acyl tryptamide to caffeine is from 1:1200 to 200:1. In another embodiment of the invention, the ratio of long chain fatty acyl tryptamide to caffeine ranges from 1:800 to 150:1. In a different embodiment of the invention, the ratio of long chain fatty acyl tryptamide to caffeine ranges from 1:400 to 100:1. In yet another embodiment of the invention, the ratio of long chain fatty acyl tryptamide to caffeine ranges from 1:200 to 50:1. In a further embodiment of the invention, the ratio of long chain fatty acyl tryptamide to caffeine ranges from 1:100 to 20:1. In a different embodiment of the invention, the ratio of long chain fatty acyl tryptamide to caffeine ranges from 1:50 to 10:1. In another embodiment of the invention, the ratio of long chain fatty acyl tryptamide to caffeine ranges from 1:20 to 5:1. In a further embodiment of the invention, the ratio of long chain fatty acyl tryptamide to caffeine ranges from 1:15 to 2:1. In yet another embodiment of the invention, the ratio of long chain fatty acyl tryptamide to caffeine ranges from 1:1 to 1:10.

While elevated quantities of tryptamides may provide improved neuroprotective performance, the caffeine does not need to be present in the composition in an elevated quantity. The composition may contain quantities of caffeine that are lower than that which is found in natural materials.

According to another embodiment, the active ingredients of the composition are the long chain fatty acyl tryptamide and caffeine. However, the composition of the invention may contain other things. According to one embodiment of the invention, the composition contains caffeine, the long chain fatty acyl tryptamide, and either a pharmaceutically acceptable carrier, excipient, electrolyte, legal stimulant, vitamin, mineral, or health supplement, or a combination thereof.

According to another embodiment of the invention, the pharmaceutically acceptable carrier may be liposomes, polymeric micelles, microspheres, nanostructures, nanofibers, dendrimers, or combinations thereof.

According to a different embodiment of the invention, the pharmaceutically acceptable excipient may be microcrystalline cellulose, dicalcium phosphate, stearic acid, magnesium stearate, croscarmellose sodium, silicon dioxide, enteric coating, natural flavors, gelatin, titanium dioxide, white rice flour, salt, acetic acid, disodium EDTA, rice bran oil, vegetable wax, gelatin, glycerin, water, colors, cellulose, pharmaceutical glaze, starch, maltodextrin, vegetable cellulose, sunflower lecithin, safflower oil, glycerin, sunflower lecithin, sorbitol, modified food starch, or combinations thereof.

According to another embodiment of the invention, the pharmaceutically acceptable electrolytes include, for example, sodium chloride, potassium, calcium, sodium bicarbonate, and the like, or combinations thereof.

According to yet another embodiment of the invention, the pharmaceutically acceptable legal stimulants include, for example, guarana, taurine, ginseng, vitamin B complex (including, for example, thiamine (vitamin B₁), riboflavin (vitamin B₂), niacin (vitamin B₃), pantothenic acid (vitamin B₅), pyridoxine (vitamin B₆), inositol (vitamin B₈) and cyanocobalamin (vitamin B₁₂), and the like, or combinations thereof.

According to a further embodiment of the invention, the pharmaceutically acceptable vitamins include, for example, vitamin A, vitamin B₁, vitamin B₂, vitamin B₃, vitamin B₅, vitamin B₆, vitamin B₇, vitamin B₉ (folic acid or folate), vitamin B₁₂, vitamin C, vitamin D, vitamin E, vitamin K, and the like.

According to a different embodiment of the invention, the pharmaceutically acceptable minerals include, for example, sodium, potassium, chloride, calcium, phosphate, sulfate, magnesium, iron, copper, zinc, manganese, iodine, selenium, molybdenum, and the like, or combinations thereof.

According to another embodiment of the invention, the pharmaceutically acceptable food and health supplements include, for example, N-acetyl L-cysteine, acetyl L-carnitine, S-adesnosyl methionine, vinpocetine, huperzine A, L-theanine, phosphatidylserine, bacopa, pterostilbene, L-tyrosine, L-glutamine, bacopin, L-pyroglutamic acid, phosphatidylserine, docosahexaenoic acid, choline, inositol, N-acetyltyrosine, gamma-aminobutyric acid, activin, L-alpha glycerylphosphorylcholine, citicoline) herb parts (e.g., leaves, roots, buds, flowers, stem or the like) or herb, fruit or botanical extracts (e.g., green tea extract, bilberry fruit standardized extract, grape skin extract, guarana extract, kola nut extract, peppermint oil, tulsi extract (holy basil), green tea extract, Gingko Biloba extract, Rhodiola extract, white tea extract, black tea extract, Panax ginseng, and the like, or combinations thereof.

Yet another embodiment of the invention is a method of treating or preventing the cognitive and movement deficits of a disease, condition or disorder. A wide variety of neurodegenerative diseases, conditions or disorders, which are known to those skilled in the art, may benefit from the use of the composition and methods of the invention. Examples, of diseases, conditions or disorders that may be treated or prevented using the composition of the invention include, for example, Alzheimer's disease, Mild Cognitive Impairment, Parkinson's disease, Parkinson's disease dementia, Lewy Body Dementia, Progressive Supranuclear Palsy, Multiple System Atrophy, Corticobasal Degeneration, Frontotemporal Dementia, Huntington's disease, Amyotrophic Lateral Sclerosis, Spinocerebellar Ataxia, Friedrich's Ataxia, bipolar disorder, cerebrovascular disorder, traumatic brain injury, encephalopathy, traumatic brain injury, Chronic Traumatic Encephalopathy, multiple sclerosis, other demyelinating and inflammatory disorders of the nervous system, and the like. In this embodiment, the composition of the invention may be administered to a subject in need of it. The composition may be administered at a dosage of at least 0.05 mg of the long chain fatty acyl tryptamide, and at least 1.5 mg of the caffeine. According to a preferred embodiment of the invention, the dosage of the long chain fatty acyl tryptamide is at least 0.5 mg, and the dosage of caffeine is at least 10 mg. According to another embodiment, the ratio of long chain fatty acyl tryptamide to caffeine used in the method may be from 1:1200 to 200:1. In a preferred embodiment, the ratio may be from 1:3 to 1:150. In yet another preferred embodiment of the invention the ratio of long chain fatty acyl tryptamide to caffeine used in the method may be from 1:5 to 1:40. In a further preferred embodiment of the invention the ratio of long chain fatty acyl tryptamide to caffeine used in the method may be from 1:6.5 to 1:30. In one embodiment, the long chain fatty acyl tryptamide used in the method may be eicosanoyl-5-hydroxytryptamide.

The neuroprotective composition of the invention may be administered by any suitable method, which methods are known to those skilled in the art. Likewise, the composition may be administered in any suitable form, which forms are known to those skilled in the art. In one embodiment of the invention, the composition may be in the form of a beverage, foodstuff, chewing gum, candy, chocolate bar, pharmaceutical composition, vitamin, nutraceutical or nutritional supplement, and the like. According to another embodiment, the beverage used in the method may be water, a fruit drink, tea, energy drink, nutritional drink or sport drink, and the like. According to yet another embodiment, the pharmaceutical composition may be administered in the form of a powder, tablet, capsule, lozenge, strips, syrup, suspension, emulsion, tincture, elixir or effervescent formulation, and the like.

Still another embodiment of the invention is a method of preventing or improving a neurological deterioration in a subject. In this embodiment, the composition of the invention is administered to a subject in need of it. The composition may be administered at a dosage of at least 10 mg of the long chain fatty acyl tryptamide, and at least 10 mg of the caffeine. Examples of neurological deterioration that may be improved or prevented by the use of the method include, for example, decline in memory, mild cognitive impairment, dementia, reduced alertness, slow movements, Parkinsonian signs, tremor, poor coordination of movements, anosmia, REM sleep behavior disorder, or any genetic locus identified as a risk factor for neurodegenerative disease, and the like.

A further embodiment of the invention is a method of reducing α-synuclein aggregation and/or tau protein aggregation in the central nervous system tissue of a subject. The method involves administering the composition of the invention to a subject in need of it.

Still a further embodiment of the invention is a method of reducing phosphorylated α-synuclein aggregate levels and/or phosphorylated tau protein levels in the central and/or peripheral tissues of a subject. In this method, the composition of the invention is administered to a subject in need of it.

Yet another embodiment of the invention is a method, according to any one of other above-mentioned methods, where the tissue of the subject being treated has at least one of the following pathologies: Lewy bodies, Lewy neurites, neurofibrillary tangles, amyloid plaques, or other pathologic protein aggregates or inclusions, and the like.

A different embodiment of the invention is a method of reducing the levels of inflammatory markers in a subject in need of such reduction, by administering the composition of any one of the embodiments of the invention. According to one embodiment, the inflammatory markers are representative of at least one of microgliosis (for example Iba-1) or astocytosis (for example GFAP).

Another embodiment of the invention is a method of increasing the levels of dopamine in a subject in need of such increase, by administering the composition of the invention.

A different embodiment of the invention is a method of increasing the tyrosine hydrolase (TH) positive dopaminergic neurons in a subject in need of such increase, by administering the composition of the invention.

Yet another embodiment of the invention is a method of increasing the levels of methylated protein phosphatase 2A (PP2A) in a subject in need of such increase, by administering the composition of the invention.

Still another embodiment of the invention is a method of decreasing the levels of demethylated protein phosphatase 2A (PP2A) in a subject in need of such a decrease, by administering the composition of the invention.

### Examples

Some embodiments of the invention will now be described in detail in the following examples. These examples are not intended to limit the scope of what the inventors regard as their invention, nor are they intended to represent that the experiments below are all, or the only experiments performed. Data are presented as means±standard error of the mean (SEM). Statistical differences among means were analyzed by one-way analysis of variance (ANOVA) followed by Newman-Keuls multiple comparison test. Statistical significance was set at p<0.05.

### 1. EHT and Caffeine Co-Treatment Effect on Behavioral Deficits of Syn^{Tg} Mice

To test for a synergistic effect of EHT and caffeine (CAF) on α-synuclein-mediated pathology, a relatively small dose of EHT in this study (12 mg/kg/day in chow) was chosen. This is the smaller of the two doses used in Syn^{Tg} mice that resulted in partial amelioration of molecular, histochemical, and behavioral benefits after nine months of treatment (Lee K W, et al. (2011) Enhanced phosphatase activity attenuates alpha-synucleinopathy in a mouse model. J Neurosci 31(19):6963-6971). For caffeine, a dose of 50 mg/kg/day in drinking water was selected based on extrapolations from the use of caffeine in the MPTP model (Chen J F, et al. (2001) Neuroprotection by caffeine and A(2A) adenosine receptor inactivation in a model of Parkinson's disease. J Neurosci 21(10):RC143; see also, Xu K, et al. (2006) Estrogen prevents neuroprotection by caffeine in the mouse 1-methyl-4-phenyl-1,2,3,6-tetrahydropyridine model of Parkinson's disease. J Neurosci 26(2):535-541) and internally generated preliminary data. The dose was determined based on one third of the dose that showed a protective effect in mice injected in the striatum with A53T mutant α-synuclein fibrils (Luan Y, et al. (2018) Chronic Caffeine Treatment Protects Against alpha-Synucleinopathy by Reestablishing Autophagy Activity in the Mouse Striatum. Front Neurosci 12:301).

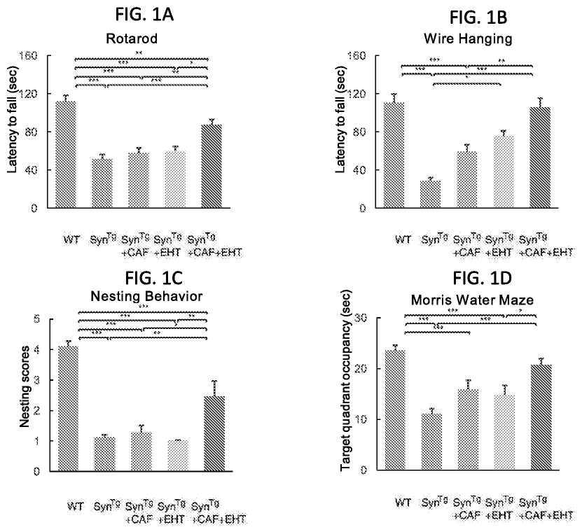

In the present study, Syn^{Tg} mice were treated with caffeine and/or EHT starting upon weaning until six months of age and were tested using four behavioral assessments that reflect the functions of different brain regions. Motor performance was evaluated on the rotarod and the Wire Hang test, which are controlled by the nigrostriatal pathway (FIG. 1, A and B) (Rozas G & Labandeira Garcia J L (1997) Drug-free evaluation of rat models of parkinsonism and nigral grafts using a new automated rotarod test. Brain Res 749(2):188-199; and also, Perez F A & Palmiter R D (2005) Parkin-deficient mice are not a robust model of parkinsonism. Proc Natl Acad Sci USA 102(6):2174-2179); nesting behavior also reflects nigrostriatal function (FIG. 1C) (Sedelis M, et al. (2000) MPTP susceptibility in the mouse: behavioral, neurochemical, and histological analysis of gender and strain differences. Behav Genet 30(3):171-182); and the Morris Water Maze test, which measures spatial learning and memory, reflects hippocampal function (FIG. 1D) (Bennett M C & Rose G M (1992) Chronic sodium azide treatment impairs learning of the Morris water maze task. Behav Neural Biol 58(1):72-75).

The results indicated that the performance of Syn^{Tg} mice was impaired on all four behavioral tests compared with wild-type (WT) mice (FIG. 1A-D). Caffeine (CAF) treatment alone in Syn^{Tg} mice did not show any improvement on any of the tests compared to untreated Syn^{Tg} mice. EHT treatment alone in Syn^{Tg} mice did not improve performance on the rotarod, nesting behavior or Morris Water Maze, but did so only on the Wire Hang test compared to untreated Syn^{Tg} mice (FIG. 1B). On the other hand, co-treatment with both CAF and EHT improved behavioral performance of Syn^{Tg} mice compared to untreated Syn^{Tg} mice and compared to those treated with one compound on some of the tests (FIG. 1, A-D). Accordingly, the combination of CAF and EHT provides a synergistic therapeutic effect at doses that are individually ineffective, yet, when in combination prevents the behavioral deficits of Syn^{Tg} mice.

2. Co-Treatment with EHT and Caffeine Reduces the Accumulation of Phosphorylated α-Synuclein Burden and Protects Against the Neuronal Damage and Neuroinflammation in Syn^{Tg} Mice.

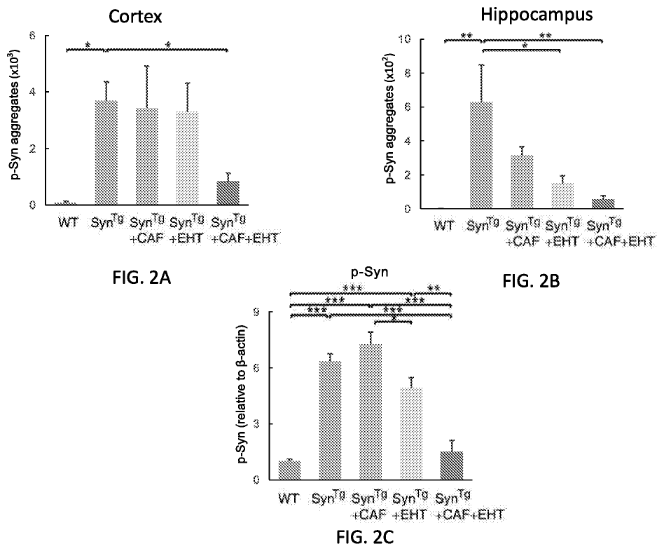

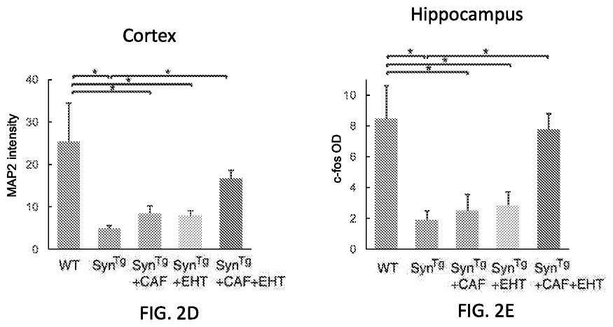

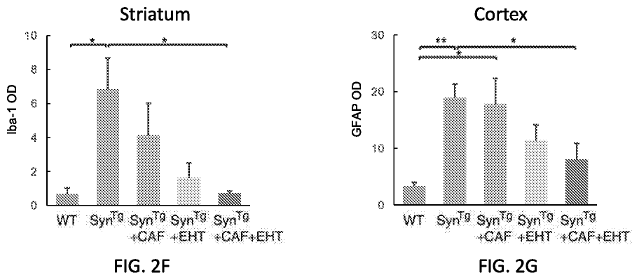

To investigate the effect of the treatments on α-synuclein phosphorylation, immunohistochemical stains for phosphorylated α-synuclein (p-α-Syn) were carried out. The results indicated that the brains from Syn^{Tg} mice had markedly increased p-α-Syn immunoreactivity in both the cerebral cortex and hippocampus compared to wild-type (WT) mice (FIG. 2, A and B). Caffeine treatment alone had no significant effect on the number of p-α-Syn immunoreactive cells compared with untreated Syn^{Tg} mice (FIG. 2, A and B), while EHT treatment alone reduced p-α-Syn positive cells in the hippocampus (FIG. 2B) but not striatum (FIG. 2A).

On the other hand, co-treatment with both EHT and CAF markedly reduced the number of p-α-Syn immunoreactive cells in both brain regions compared with untreated Syn^{Tg} mice (FIG. 2, A and B) supporting a synergistic impact upon co-administration of EHT and CAF. A similar profile of changes in p-α-Syn levels was observed using Western blot analysis with cortical tissue lysates from five animals in each group (FIG. 2C). Consistent with immunohistochemistry, Western blots showed a six-fold increase in p-α-Syn levels in Syn^{Tg} mice compared to WT mice (FIG. 2C). Caffeine or EHT treatments separately had no significant effect compared to untreated Syn^{Tg} mice, whereas co-treatment with both compounds reduced p-α-Syn levels down to WT levels.

The integrity of neuronal structure and activity were next assessed in the five groups of mice. Syn^{Tg} mice have substantial depletion of the cytoskeletal microtubule associated protein 2, MAP2, in the cortex (FIG. 2D), suggestive of reduced dendritic complexity (Harada A, Teng J, Takei Y, Oguchi K, & Hirokawa N (2002) MAP2 is required for dendrite elongation, PKA anchoring in dendrites, and proper PKA signal transduction. J Cell Biol 158(3):541-549). Administration of caffeine or EHT alone did not improve this profile, whereas co-treatment with both compounds restored neuritic integrity. A similar profile was observed for the immunoreactivity to the immediate early gene product c-fos in the hippocampus, a marker used as a surrogate of neuronal activity (Palop J J, et al. (2003) Neuronal depletion of calcium-dependent proteins in the dentate gyrus is tightly linked to Alzheimer's disease-related cognitive deficits. Proc Natl Acad Sci USA 100(16):9572-9577). Compared with WT mice, Syn^{Tg} mice showed a marked loss of c-fos expression (FIG. 2E). Treatment of Syn^{Tg} mice with either caffeine or EHT failed to increase c-fos immunoreactivity significantly, but the combination preserved this marker to near WT levels (FIG. 2E). The latter finding is consistent with the improved performance of Syn^{Tg} mice given both compounds on the Morris Water Maze test (FIG. 1D).

Neuroinflammation is one of the neuropathological features of PD and models of α-synucleinopathy including Syn^{Tg} mice (Lee K W, et al. (2011) Enhanced phosphatase activity attenuates alpha-synucleinopathy in a mouse model. J Neurosci 31(19):6963-6971). In this study, it was also observed that untreated Syn^{Tg} mice exhibited marked microglial activation (ionized calcium-binding adapter molecule 1, Iba1) in the striatum, and astrocytic proliferation (glial fibrillary acidic protein, GFAP) in the cortex, that were partially but insignificantly attenuated by caffeine or EHT treatment each given alone. However, the reduction of these markers was significant with the combined administration of both compounds (FIG. 2, F and G). These findings confirm that the co-treatment has a synergistic effect in protecting neuronal integrity and preventing the inflammatory response to the α-synuclein transgene in these mice.

3. Co-Treatment with EHT and Caffeine Improves the Behavioral Performance of α-Syn PFF Inoculated WT Mice.

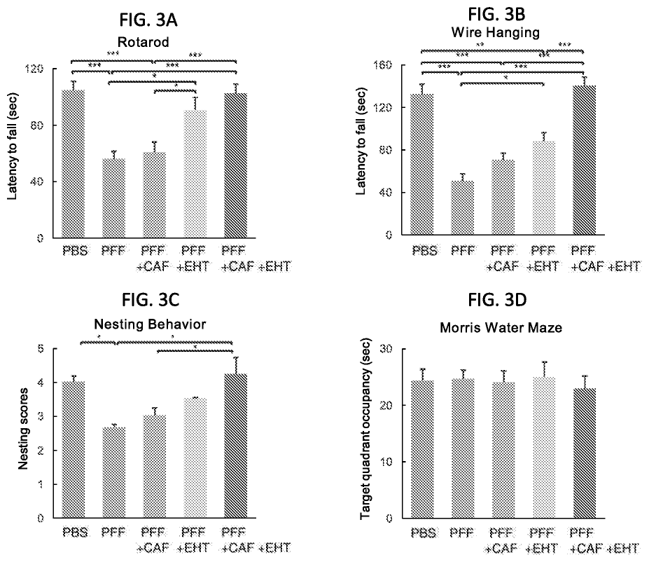

The effect of EHT and/or caffeine was also tested in a second model of α-synucleinopathy, in which mouse α-Syn PFF injected in the dorsal striatum nucleate endogenous α-synuclein and propagate to anatomically linked brain regions including nigral dopaminergic neurons (Luk K C, et al. (2012) Pathological alpha-synuclein transmission initiates Parkinson-like neurodegeneration in nontransgenic mice. Science 338(6109):949-953). WT mice were placed upon weaning on a diet containing 12 mg/kg/day EHT (or control diet), or given 50 mg/kg/day caffeine in drinking water (or normal water), or the two combined. These are the same doses used in Syn^{Tg} mice described above. Animals were then injected at two months of age with α-Syn PFF or PBS in the right striatum to induce PD-like pathology. Six months later (at 8 months of age), behavioral tests were performed as described above. Untreated mice inoculated with α-Syn PFF showed significantly impaired performance in three behavioral tests (rotarod, Wire Hang and nesting behavior) that are related to nigrostriatal function (FIG. 3, A-C). Caffeine treatment alone in α-Syn PFF injected mice did not affect performance on any of the tests. EHT given alone improved performance on the rotarod and wire hang test but not nest building.

On the other hand, the combination treatment improved performance on all three tests that were impacted by α-Syn PFF inoculation (FIG. 3, A-C). Learning and memory task on the Morris Water Maze, a test of hippocampal function, is not affected by α-Syn PFF inoculation or the treatments (FIG. 3D). These findings suggest that the co-administration of EHT and caffeine protects against the behavioral deficits in the α-Syn PFF model significantly better than each treatment alone.

4. Co-Treatment with EHT and Caffeine Prevents the Nucleation and Propagation of p-α-Syn Positive Aggregates, Mitigates Neuroinflammation, and Protects Nigrostriatal Neurons in the α-Syn PFF Inoculation Model.

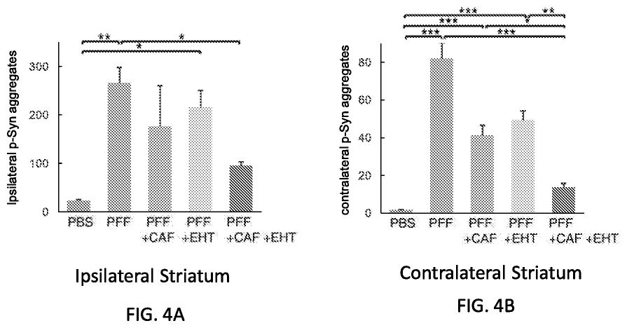

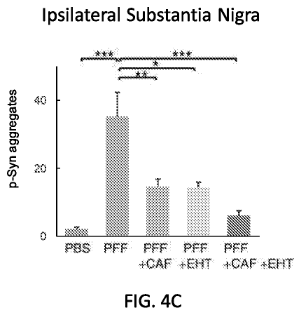

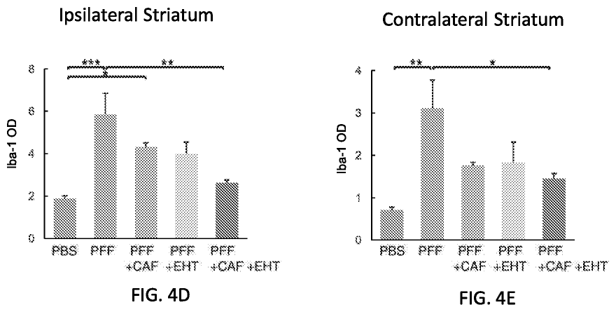

Following completion of behavioral assessments, mice were sacrificed and their brains examined for p-α-Syn immunoreactivity, neuroinflammation and the integrity of the nigrostriatal pathway. p-α-Syn in both the ipsilateral and contralateral striata was partially but insignificantly reduced with each of caffeine or EHT treatment given separately compared to untreated α-Syn PFF inoculated mice, but the reduction with the combination treatment was significant (FIG. 4, A and B). Aggregates in the contralateral striatum were less abundant in co-treated animals compared with inoculated mice treated with each compound separately. In the ipsilateral nigra, p-α-Syn immunoreactivity was less abundant in all three treatment groups, but this effect was more pronounced in mice treated with both compounds (FIG. 4C). These findings suggest a synergistic effect of EHT and CAF in preventing the seeding and propagation of α-Syn PFF induced pathologic aggregates.

α-Syn PFF inoculation induced microglial activation in the ipsilateral and contralateral striata (FIG. 4, D and E) as described previously (Blumenstock S, et al. (2017) Seeding and transgenic overexpression of alpha-synuclein triggers dendritic spine pathology in the neocortex. EMBO Mol Med 9(5):716-731; and Boza-Serrano A, et al. (2014) The role of Galectin-3 in alpha-synuclein-induced microglial activation. Acta Neuropathol Commun 2:156)). The optical density (OD) of the Iba-1 signal in the ipsilateral striatum was about two fold higher than that in the contralateral side. Compared with untreated α-Syn PFF inoculated group, the prevention of microglial activation was significant in both striata only in mice co-treated with EHT and CAF, whereas each compound administered separately had only a partial effect that was statistically insignificant.

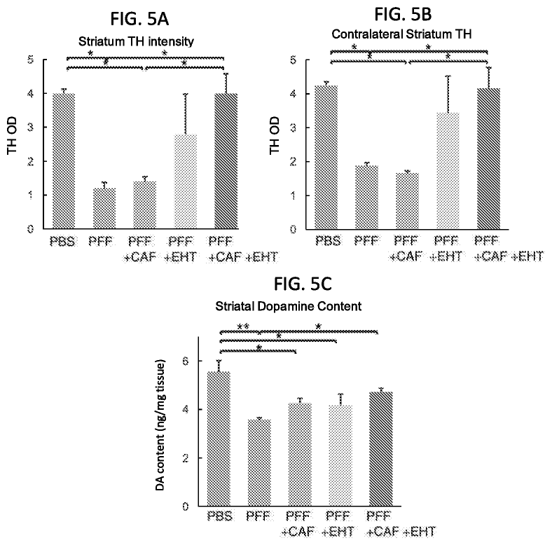

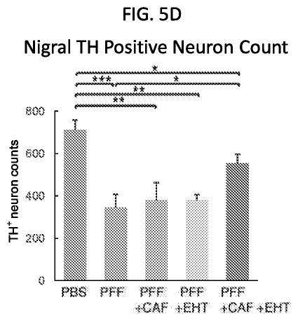

Assessment of the integrity of the nigrostriatal pathway revealed a similar profile (FIG. 5 A-D). Tyrosine hydroxylase (TH) staining of dopaminergic terminals in the striatum revealed bilateral depletion in untreated α-Syn PFF inoculated mice compared with PBS injected animals. Caffeine alone had no effect on the decline of this marker, and EHT alone was associated with a non-significant increase compared to untreated α-Syn PFF inoculated mice. The combination treatment, however, resulted in a significantly greater preservation of TH positive terminals when compared with untreated α-Syn PFF inoculated mice (FIG. 5A, B).

Similarly, dopamine (DA) content in lysates of the ipsilateral striatum, measured by high-performance liquid chromatography-mass spectrometry (HPLC-MS), was depleted by 36% in the α-Syn PFF inoculated group compared with the PBS injected group (FIG. 5C). Co-treatment with EHT and caffeine preserved dopamine content by 32% compared with untreated α-Syn PFF inoculated mice, while treatment with each compound alone did not have a significant benefit.

TH positive dopaminergic neurons of the substantia nigra showed a similar profile whereby α-Syn PFF inoculation reduced the number of these neurons by 51% compared to PBS injected mice (FIG. 5D). EHT or caffeine treatment individually did not prevent this reduction. However, co-treatment with EHT and caffeine was associated with only 22% reduction compared to PBS injected mice, representing a 59% protection compared to untreated α-Syn PFF inoculated group.

5. EHT and Caffeine Exert their Synergistic Neuroprotective Effects Through Regulating PP2A Activity.

EHT was identified and purified because of its ability to inhibit the activity of the PP2A methylesterase (PME-1) leading to enhanced methylation and activity of PP2A. Considering the synergy between caffeine and EHT in the behavioral and neurochemical profiles detailed above, the two compounds were tested to determine if they also synergize their respective activities in modulating PP2A methylation. As PP2A is relatively demethylated in postmortem brains of α-synucleinopathy cases, including PD and Dementia with Lewy Bodies (Park et al 2016, Dysregulation of protein phosphatase 2A in Parkinson disease and dementia with lewy bodies. Ann Clin Transl Neurol. 2016 Sep. 7; 3(10):769-780), the inventors further looked to determine whether PP2A methylation changes also occur with α-synuclein over-expression and α-Syn PFF challenge in mice.

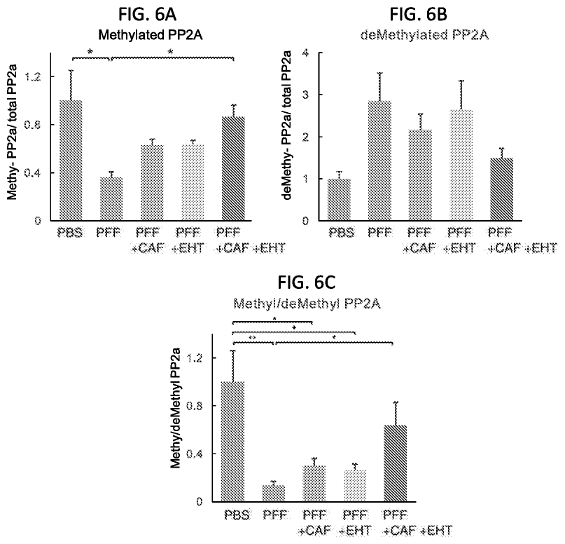

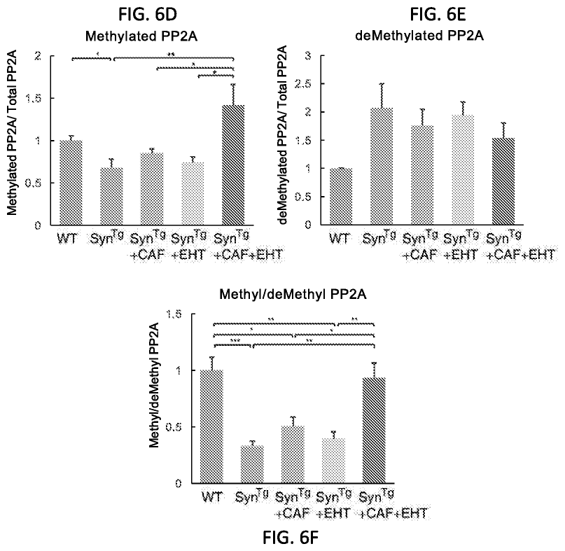

The state of PP2A methylation in the brains of Syn^{Tg} mice without or with EHT and/or caffeine treatment was assessed by Western blot analysis. Methylated PP2A (Methyl-PP2A) levels were lower in untreated Syn^{Tg} mice compared with WT mice, did not change with either caffeine or EHT administration given separately, but increased with the combination treatment (FIG. 6 A and B). Demethylated PP2A (demethyl-PP2A) tended to be higher in Syn^{Tg} mice compared with WT mice, and was not affected by any of the treatments (FIG. 6, A and C). However, the ratio between methyl- and demethyl-PP2A was markedly lower in Syn^{Tg} mice compared with WT mice, did not change by EHT or CAF treatments alone, but was significantly maintained at WT levels by co-administration of both compounds (FIG. 6D).

The results indicated that in striatal tissue lysates following α-Syn PFF inoculation, methyl-PP2A levels as well as the methyl- to demethyl-PP2A ratio were reduced compared with PBS injected striata (FIG. 6, E-H). Caffeine and EHT treatment given separately to these mice resulted in an insignificant increase in these measures compared with untreated α-Syn PFF inoculated mice, but the effect of the combination treatment was significant. Thus, this profile of PP2A methylation changes is consistent with that seen in Syn^{Tg} mice given these treatments (FIG. 6, A-D).

### 6. Combination of EHT and Caffeine has Synergistic Effects in Up-Regulating PP2A Methylation and Attenuating Cytotoxicity Induced by α-Syn PFF in SH-SY5Y Cells.

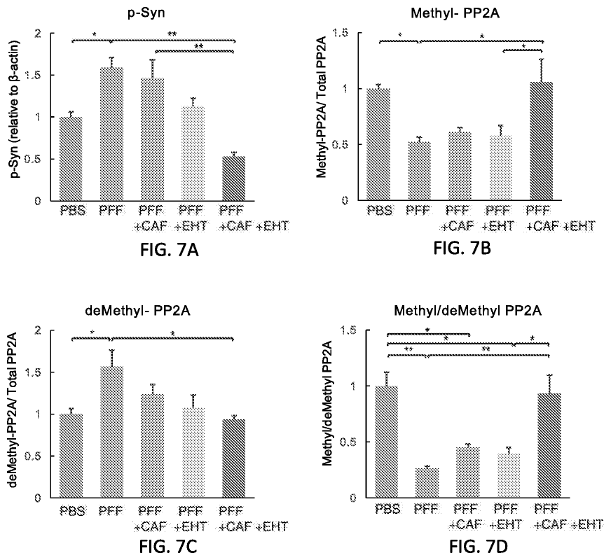

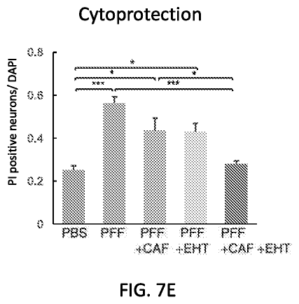

The above in vivo findings were further confirmed in an analogous experiment with human neuroblastoma SH-SY5Y cells (FIG. 7 A-E). Challenging these cells with mouse α-Syn PFF reduced methylated PP2A levels with a reciprocal increase in demethylated PP2A levels (FIG. 7 C and D). Adding either caffeine or EHT to the medium partially reversed this trend, but the combination fully corrected these PP2A methylation changes to levels seen in PBS treated cells. As a result, methyl- to demethyl-PP2A ratio, which was markedly reduced by α-Syn PFF challenge, was completely restored by EHT and CAF added together to the culture medium but not individually (FIG. 7E). These alterations in PP2A methylation profile were associated with parallel changes in p-α-Syn levels (FIG. 7B). Challenging cells with α-Syn PFF increased p-α-Syn levels, caffeine alone had no effect, EHT alone reduced it partially, but the combination of both compounds had a much bigger effect.

Furthermore, the cytotoxicity of α-Syn PFF, assessed by propidium iodide (PI) exclusion, reflected a similar profile (FIG. 7F). The number of PI positive cells increased 2.5 fold with α-Syn PFF challenge, was modestly protected by caffeine and EHT added to the culture medium separately, but the combination of both compounds improved cell viability significantly compared to untreated α-Syn PFF-challenged cells. These findings support the hypothesis that EHT and caffeine can act in synergy through enhancing steady state levels of PP2A methylation, and hence phosphatase activity, associated with cytoprotection.

The present invention is directed to the new discovery that subtherapeutic doses of caffeine and EHT, two unrelated compounds found in coffee, can work in synergy to effect biochemical and molecular changes in the mouse brain leading to protection in two models of α-synucleinopathy. This is reflected in better behavioral performance of both Syn^{Tg} mice and α-Syn PFF inoculated mice treated chronically with a combination of these compounds for six months but not if each is given separately. In addition to preserved neuronal integrity and function, and dampened inflammatory response to α-synuclein, PP2A methylation is modulated by this co-treatment in a manner that favors enhanced phosphatase activity. This is associated with reduced accumulation of p-α-Syn Similar biochemical changes occur in a cellular model challenged with α-Syn PFF and treated with the combination leading to increased PP2A methylation, reduced p-α-Syn levels, and cytoprotection.

These observations collectively confirm that caffeine and EHT may function through a common new mechanism. In this study, EHT was purified from coffee in an analytical assay set up to identify specifically compounds that inhibit the PP2A methylesterase PME-1 (Lee K W, et al. (2011) Enhanced phosphatase activity attenuates alpha-synucleinopathy in a mouse model. J Neurosci 31(19):6963-6971). EHT administered alone to Syn^{Tg} mice for nine months also modulated PP2A methylation in brain tissue lysates in a dose dependent manner in favor of the enzymatically active form and reduced the accumulation of p-α-Syn. This was more evident at a dose ten times higher (120 mg/kg/day) than the dose used in the present study.

Caffeine, on the other hand, is an adenosine A2a receptor antagonist, a property that is believed to mediate its protective function in the MPTP model of dopamine neuron degeneration. In addition, deleting the A2a receptor gene in mice has been shown to protect against dopaminergic neuron degeneration induced by human α-synuclein transgene containing two pathogenic mutations, A53T and A30P (Kachroo A & Schwarzschild M A (2012) Adenosine A2A receptor gene disruption protects in an alpha-synuclein model of Parkinson's disease. Ann Neurol 71(2):278-282). Caffeine was also recently reported to protect against A53T mutant α-synuclein fibril injections in the striatum (Luan Y, et al. (2018) Chronic Caffeine Treatment Protects Against alpha-Synucleinopathy by Reestablishing Autophagy Activity in the Mouse Striatum. Front Neurosci 12:301). By blocking A2a receptor signaling, caffeine may enhance PP2A methylation through preventing cAMP dependent protein kinase A (PKA)/glycogen synthase kinase 3β (GSK3β) mediated activation of PME-1.

The combined effect of EHT and caffeine is greater PP2A activity than either compound could achieve alone. The latter could underlie the synergy with EHT observed in the present study. Thus, these results suggest that chronic consumption of coffee and, therefore, chronic co-ingestion of both EHT and caffeine, have added benefits in α-synucleinopathy prone brains. Additionally, the increase in methylated PP2A with these treatments associated with neuroprotection, along with hypomethylation of PP2A in PD and DLB brains suggests a pathogenetic significance of this phosphatase in these disorders.

The protection in the α-Syn PFF model with caffeine and EHT co-administration provides some insight into the role of α-synuclein phosphorylation in nucleation and propagation of pathologic aggregates. This treatment was initiated upon weaning of wild-type mice, and α-Syn PFF inoculation occurred six weeks later. Thus, the reduced phosphorylation level of α-synuclein in the brain appears to have decreased the ability of exogenous fibrils to nucleate endogenous α-synuclein at the site of striatal injection leading to reduced propagation of aggregates to the substantia nigra pars compacta and the contralateral striatum.

Methods and Methods:

### Animals:

Thy1-α-synuclein transgenic mice (Syn^{Tg}) on BDF1 background overexpressing human wild-type α-synuclein under the control of the Thy1 promoter were maintained by mating heterozygous transgenic females with WT BDF1 males (Rockenstein E, et al. (2002) Differential neuropathological alterations in transgenic mice expressing alpha-synuclein from the platelet-derived growth factor and Thy-1 promoters. J Neurosci Res 68(5):568-578). BDF1 mice (mixed C57BL/6-DBA/2) were generated every three months by mating female C57BL/6 (The Jackson Laboratory) and male DBA/2 mice (Charles River). Experiments were performed with male transgenic mice and their WT littermates. Upon weaning, Syn^{Tg} mice were placed either on regular drinking water or water containing caffeine (Sigma-Aldrich) to deliver 50 mg/kg/day, and/or either control mouse chow or chow containing eicosanoyl-5-hydroxytryptamide (EHT) to deliver 12 mg/kg/day. Wild-type littermates received regular water and control chow. For α-Syn PFF injection model, C57BL/6J male mice were placed upon weaning either on regular drinking water or water containing caffeine (50 mg/kg/day), and/or either control chow or chow containing EHT (12 mg/kg/day). At two months of age, mice were inoculated with α-Syn PFF unilaterally in the striatum. These treatments continued throughout the experiment until animals were sacrificed. Mice were weighed and their food and water intake was quantified weekly. Animals were housed 2-5/cage in an AAALAC approved facility in a temperature- and humidity-controlled environment under a 12-hour light/dark cycle and were maintained on a diet of lab chow and water ad libitum. All animal procedures were approved by the Rutgers-Robert Wood Johnson Medical School Institutional Animal Care and Use Committee and were performed according to the National Institutes of Health Guide for the Care and Use of Laboratory Animals.

Reagents:

The following reagents were used during testing:

EHT synthesized at Signum Biosciences (Princeton, N.J.).
Caffeine purchased from Sigma-Aldrich.
Primary antibodies used were: anti-phospho-Ser129-α-synuclein (#015-25191, WAKO), anti-MAP2 (ab32454, Abcam), anti-c-fos (sc-52, Santa Cruz), anti-Iba1 (#019-19741, WAKO), anti-GFAP (GA524, Dako), anti-tyrosine hydroxylase (TH) (T2928, Sigma), anti-methylated PP2A (clone 4D9, generated at Princeton University), anti-demethylated PP2A (#05-577, Millipore), anti-total PP2A (ab32065, Abcam), anti-β-actin (A5441, Sigma) and anti-β-tubulin (#T8328, Sigma). Secondary antibodies used are as follows: IRDye 800CW anti-mouse IgG (925-32210, Li-Cor), IRDye 800CW anti-rabbit IgG (925-32211, Li-Cor), HRP conjugated anti-mouse IgG (HAF007, R&D systems), HRP conjugated anti-rabbit IgG (HAF008, R&D systems), FITC conjugated anti-rabbit IgG (F9887, Sigma).

Synuclein Preformed Fibril (PFF) Preparation:

Plasmid (pT7-7) encoding mouse α-synuclein cDNA (Weinreb P H, et al. (1996) NACP, a protein implicated in Alzheimer's disease and learning, is natively unfolded. Biochemistry 35(43):13709-13715) was used to transform Escherichia coli BL21(DE3) strain (Invitrogen Inc.). One liter of LB with transformed E coli culture was incubated at 37° C. When the OD600 reading reached 0.8, expression of α-synuclein was induced by adding 1 mL of 1M isopropyl β-D-1-thiogalactopyranoside. Culture was incubated further for 4 hours, and then cells were collected by centrifugation at 2,000 g for 30 minutes. Cell pellets were collected and resuspended in 25 ml phosphate buffered saline (PBS). Subsequently, cells were homogenized 3 times by Emulsiflex C5 Homogenizer (AVESTIN). Lysate was then centrifuged at 24,000 g for 30 minutes. Supernatant was collected and 10 mg/mL streptomycin sulfate was added. Solution was stirred at 4° C. for 30 minutes and then centrifuged at 24,000 g for 30 minutes. Supernatant was collected again and 0.361 g/mL ammonium sulfate was added. Sample was stirred at 4° C. for 1 hour and then centrifuged at 24,000 g for 30 minutes. The pellet was collected and resuspended in 15 ml PBS and then boiled in a water bath for 15 minutes. After cooling, the sample was centrifuged at 24,000 g for 30 minutes, and the supernatant was collected and dialyzed into 25 mM Tris buffer pH 7.7. α-Synuclein containing solution was then separated by fast protein liquid chromatography (FPLC, GE healthcare) using 5 mL Anion exchange column Hitrap Q (GE healthcare) and eluted with NaCl (30%, 50% and 100% gradient). Solutions containing α-synuclein were dialyzed against ammonium bicarbonate before lyophilization, and the freeze-dried α-synuclein was dissolved in PBS at 5 mg/mL. α-Synuclein solution was then subjected to shaking at 1000 rpm at 37° C. for 7 days on a thermomixer C (Eppendorf). Formation of fibrillar α-synuclein was monitored and confirmed by thioflavin-T assay.

Stereotaxic Inoculation of α-Syn PFF

α-Syn PFF solution was sonicated at 60% amplitude 30 times (0.5 sec on, 0.5 sec off). During the stereotactic surgery, two month old WT C57BL/6 mice were anesthetized by Ketamine/xylazine (90/4.5 mg/kg, i.p., Ketaset from Zoetis, and Anased from Akorn) and positioned in a digital stereotaxic apparatus (Stoelting, Wood Dale, Ill.). A midline sagittal incision was made in the scalp, and a hole was drilled in the skull over the right striatum according to the coordinates below. All injections were made using a Hamilton neuros syringe equipped with a 33 gauge needle and attached to a Quintessential Stereotaxic Injector (Stoelting, Wood Dale, Ill.). A total of 5∝g/2.5 ul of α-Syn PFF was infused in the dorsal striatum (+0.2 mm relative to Bregma, +2.0 mm from midline, +2.6 mm beneath the dura) at a delivery rate of 0.2∝l/min (61). The needle was kept in place for 5 min following the infusion to prevent reflux before withdrawing.

Behavioral Tests:

Behavioral tests were conducted at six months of age in Syn^{Tg} mice treated with EHT and/or caffeine and in WT mice, and at six months post α-Syn PFF- or PBS-injections in WT mice when animals were 8 months old.

Rotarod: the rotarod test was performed using the automated TSE system. Mice were placed on the rod with an accelerating speed and were trained for four trials for the first four days. The first two trials were acquisition trials where speed increases from 4 to 20 rpm during 180 seconds. The last two trials were the actual probe trials where speed increases from 4 to 40 rpm over 180 seconds. On the fifth day, mice were given the same probe trials three times, and latency of each animal to fall was recorded.

Wire Hang: this test is to measure the latency of a mouse to fall after hanging from a metal wire, 55 cm long and 2-mm thick, and linked between two vertical stands. The wire is installed 30 cm above the bedding material to prevent injury to the animal when it falls. The latency of mice to fall was measured during a maximum window of 180 seconds. The test is repeated three times with an interval of 30 minutes, and the average is used for analysis.

Morris Water Maze: all animals received a one-day pre-acquisition training where the platform was visible. Then animals were trained with a hidden platform remaining in the same position for four days. Four trials were performed each day, and mice were released from four different quadrants. On the sixth day, mice were given the probe test with the platform removed. The latency of each animal spent in the target quadrant was recorded.

Nesting Behavior: this test was performed by placing a single mouse in a new cage with a 5 cm tightly packed cotton square Nestlet (Ancare). Fifteen hours later, nest formation was recorded and then rated blindly on a scale of 1 (non-shredded) to 5 (maximally shredded) (Deacon R M (2006) Assessing nest building in mice. Nat Protoc 1(3):1117-1119).

Immunohistochemistry and Immunofluorescence

Immunohistochemical analyses were performed as described previously (Lee et al., 2011; 2012). Mice were perfused transcardially with PBS, and brains were removed and post-fixed in 10% formalin at 4° C. overnight. Fixed brains were then sectioned coronally in 30 am thickness using a cryostat and collected as sets of slices with the same interval. Free-floating sections were blocked by 1% BSA and 0.2% Triton X100 in PBS. Sections were then incubated with primary antibody overnight at 4° C. and biotinylated secondary antibody for 1 hour at room temperature.

Vectastain elite ABC kit (Vector Laboratories, Burlingame, Calif.) and 3,3′-diaminobenzidine (Sigma-Aldrich) were used for amplifying and color development. Images were captured by Nikon Eclipse 55i. Staining intensity and phosphorylated α-synuclein (p-α-Syn) aggregate counts were obtained by Image Pro Plus. Intensity calibration was set to the level of a blank area in each image. HSI (hue, saturation and intensity) was used for the color selection with the standard parameters of H: 0-30, S: 0-255 and I: 0-160. To count only positively stained cells, color selection was adjusted according to the antibody used and background intensity. Stains that were smaller than four pixels were excluded from the analysis. For striatal TH staining, an elliptical area of interest (AOI) of the same size that encompasses the striatum was applied to all the sections. For hippocampus c-fos and p-α-Syn staining, multiple AOIs of the same sizes that encompass Cornu Ammonis 2 (CA2) and CA3 regions were applied uniformly to all sections. For other stains, the whole microscopic field was analyzed. For p-α-Syn stains in the cortex, four matching regions in each of two sections per animal were counted. In the dorsal striatum, three matching regions in each of three sections per brain were counted.

In the hippocampus, three matching regions in the CA2 and CA3 per animal were counted. In the substantia nigra pars compacta (SNc), two images covering the whole SNc of each of two sections per brain were counted. For c-fos staining, two sections from each brain were analyzed. For Iba-1 staining in the striatum, four matching regions in each of four sections per brain were analyzed. For GFAP staining in the cortex, six matching regions in each of four sections per brain were analyzed.

For immunofluorescence staining, 20 gm thick cryostat sections were blocked with 5% goat serum and 0.2% Triton X-100 in PBS. Sections were then incubated with primary antibody overnight at 4° C. and fluorescent secondary antibody for 1 hour at room temperature. Images were captured using Carl Zeiss axiovert 200 microscope. For MAP2 staining in the cortex, two matching regions in each of three sections per brain were analyzed.

Striatal Dopamine Content Using HPLC-MS

Dissected striatal tissue was homogenized in 1 mL/100 mg 50% acetonitrile, 0.04M HCL and 2.7 mM EDTA water solution with 100 ng/100 mg DA.HCL-d4 as internal standard. The mixture was then sonicated for 30 seconds. Lysates were centrifuged at 6,500 g and the supernatant was filtered through 0.2 gm PTFE microfilters. Before loading to the HPLC-MS, 50 gl sample was evaporated to completely dry. Then, 20 gl 0.2% formic acid was added to resolubilize samples for 30 mins. Samples were then sonicated for 10 seconds and centrifuged at 6,500 g for five mins. HPLC-MS experiments were performed using a U3000 (Dionex) online with Velos LTQ Orbitrap Pro (ThermoFisher), but only LTQ part was used in this application. In general, 5 gL sample was injected in microliter pick up mode and separated by a reverse phase column (Discovery BIO Wide Pore C18, 5 cm×2.1 mm, Supelco analytical). Solvent A: aqueous solution of 0.5% acetic acid, solvent B: methanol was used for a gradient elution at a flow rate of 200 gl/min. The HPLC elution program was as follows: 5% B (3 min), 5-70% B (linear increase in 2 min), 70% B (5 min), 70-5% B (linear decrease in 1 min), and equilibration at 5% B (4 min). The column temperature was maintained at 45° C. The MS acquisition conditions were as follows: the electrospray ion source was operated in positive ion mode (ESI+). The positively charged DA (154 for DA and 158 for DA_d4) were isolated in ion trap with isolation window of 4 daltons and fragmented with CID with relative collision energy of 25% and activation time of 10 milliseconds for both DA and DA-d4. The fragment of 137 and 141 of DA and Da-d4 were used for quantification.

TH Immunoreactive SNc Dopamine Neuron Count:

Sections through the SNc stained for TH were scanned by Fimmic Oy (Helsinki, Finland) with Pannoramic P250 Flash II whole-slide scanner (3DHistech, Hungary) at 0.24 gm/pixel resolution. An extended focus-mode where a total depth of 58 gm was acquired as 30 focal layers with 2 gm intervals to render the whole section depth in a single focal image. Digital images were uploaded to Aiforia Cloud platform (Fimmic Oy). Automated counting of nigral TH-positive neurons was carried out using the Aiforia Cloud where a context-intelligent neural network algorithm developed specifically for counting TH-positive neurons performed an unbiased analysis. SNc sections ipsilateral to α-Syn PFF injections from five mice per group were counted using four sections from each brain at 150 gm intervals.

Cell Culture, Western Blot Analysis and Propidium Iodide (PI) Staining:

Human neuroblastoma SH-SY5Y cells were cultured in DMEM-F12 with 10% FBS. At 50% confluency, cells were treated with 1 gg/ml mouse α-Syn PFF in DMEM-F12 with 1% FBS for 7 days. Medium was refreshed every 3 days. Cell harvesting was done with 2% SDS in PBS supplemented with protease inhibitor (Millipore) and phosphatase inhibitor (Sigma-Aldrich). Lysates were sonicated and boiled at 95° C. for 5 minutes. Protein concentration was determined by bicinchonic acid (BCA) method (Pierce), equal amount of protein was separated on 5-20% SDS-PAGE gel (Genescript) and transferred to nitrocellulose or PVDF membrane (Biorad). Following transfer, membranes were blocked for 1 hour at room temperature in blocking buffer (Li-Cor) or 5% (w/v) BSA/TBS-Tween 0.1% (v/v). Primary antibodies were diluted in blocking buffer or 5% (w/v) BSA/TBS-Tween 0.1% (v/v) and incubated with the membranes at 4° C. overnight. Membranes were washed three times in TBS-Tween and incubated in diluted IRDye 800CW or HRP conjugated secondary antibody for 1 hour at room temperature. Following washing, membranes were scanned by Li-Cor Odyssey CLx infrared imaging system or treated with the Western Lightning ECL-plus reagents (PerkinElmer) and exposed to autoradiography films (LabScientific).

For PI staining, SH-SY5Y cells were incubated with 2.5 ∝M PI in serum-free medium for 5 min in a CO2 incubator. Cells were then fixed with 10% formalin for 10 min, followed by blocking with 5% goat serum and 0.2% Triton X100 in PBS for 20 min. Subsequently, cells were incubated with 0.1 ∝g/ml DAPI for 1 min and washed with PBS.

Statistical Analysis:

Data are presented as means±standard error of the mean (SEM). Statistical differences among means were analyzed by one-way analysis of variance (ANOVA) followed by Newman-Keuls multiple comparison test. Statistical significance was set at p<0.05.

It will be appreciated by persons skilled in the art that formulations described herein are not limited to what has been particularly shown and described. Rather, the scope of the formulation is defined by the claims which follow. It should further be understood that the above description is only representative of illustrative examples of embodiments. The description has not attempted to exhaustively enumerate all possible variations. The alternate embodiments may not have been presented for a specific portion of the formulation, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

1. A neuroprotective composition comprising caffeine or a caffeine analogue and a long chain fatty acyl tryptamide comprising an aliphatic chain having 16 to 22 carbons linked to a tryptamine,
wherein the composition contains from at least 1.5 mg to 600 mg of caffeine per serving or unit dosage of the composition;
wherein the composition contains from at least 0.5 mg to 300 mg of the long chain fatty acyl tryptamide per serving or unit dosage of the composition; and
wherein the ratio of long chain fatty acyl tryptamide to caffeine is from 1:20 to 2:1.

2. The composition according to claim 1, wherein the composition contains from at least 5 mg to 20 mg of caffeine per serving or unit dosage of the composition, and from at least 0.5 to 10 mg of the long chain fatty acyl tryptamide per serving or unit dosage of the composition, and the ratio of long chain fatty acyl tryptamide to caffeine is from 1:10 to 1:1.

3. The neuroprotective composition according to claim 1, wherein the long chain fatty acyl tryptamide is saturated.

4. The neuroprotective composition according to claim 1, wherein the tryptamine is a 5-hydroxytryptamine.

5. The neuroprotective composition according to claim 1, wherein the long chain fatty acyl tryptamide is eicosanoyl-5-hydroxytryptamide.

6. The neuroprotective composition according to claim 1, wherein the composition consists essentially of the caffeine, the long chain fatty acyl tryptamide, and at least one of, a pharmaceutically acceptable carrier, excipient, electrolyte, legal stimulant, vitamin, mineral, or health supplement.

7. The neuroprotective composition according to claim 5, wherein the pharmaceutically acceptable carrier is selected from the group consisting of liposomes, polymeric micelles, microspheres, nano structures, nanofibers, and dendrimers.

8. The neuroprotective composition according to claim 5, wherein the pharmaceutically acceptable excipient is selected from the group consisting of microcrystalline cellulose, dicalcium phosphate, stearic acid, magnesium stearate, croscarmellose sodium, silicon dioxide, enteric coating, natural flavors, gelatin, titanium dioxide, white rice flour, salt, acetic acid, disodium EDTA, rice bran oil, vegetable wax, gelatin, glycerin, water, colors, cellulose, water, dicalcium phosphate, pharmaceutical glaze, starch, maltodextrin, vegetable cellulose, sunflower lecithin, safflower oil, glycerin, sunflower lecithin, sorbitol, and modified food starch.

9. The neuroprotective composition according to claim 1, wherein the active ingredients of the composition consist of the long chain fatty acyl tryptamide and the caffeine.

10. The neuroprotective composition according to claim 1, wherein the long chain fatty acyl tryptamide and the caffeine are in the form of nanoparticles or microparticles.

11. The neuroprotective composition according to claim 1, wherein the nanoparticles or microparticles have a diameter of from at least 10 nm to no more than 500 nm.

12. (canceled)

13. A method of treating or prophylactically treating a patient at risk of developing cognitive and movement deficits of a disease, condition or disorder selected from the group consisting of Alzheimer's disease, Mild Cognitive Impairment, Parkinson's disease, Parkinson's disease dementia, Lewy Body Dementia, Progressive Supranuclear Palsy, Multisystem Atrophy, Corticobasal Degeneration, Frontotemporal Dementia, Huntington's disease, Amyotrophic Lateral Sclerosis, Spinocerebellar Ataxia, Friedrich's Ataxia, bipolar disorder, cerebrovascular disorder, traumatic brain injury, encephalopathy, traumatic brain injury, Chronic Traumatic Encephalopathy, multiple sclerosis, and other demyelinating and inflammatory disorders of the nervous system, comprising administering the composition of claim 1 at a dosage of at least 0.5 mg of the long chain fatty acyl tryptamide, and at least 1.5 mg of the caffeine.

14. The method according to claim 13 wherein the long chain fatty acyl tryptamide is eicosanoyl-5-hydroxytryptamide.

15. The method according to claim 13, wherein the ratio of long chain fatty acyl tryptamide to caffeine is from 1:10 to 1:1.

16. The method according to claim 13, wherein the neuroprotective composition is administered in a form selected from a beverage, foodstuff, chewing gum, candy, chocolate bar, pharmaceutical composition, nutraceutical or nutritional supplement.

17. The method according to claim 16, wherein the beverage is selected from water, a fruit drink, coffee, tea, energy drink, a nutritional drink or a sport drink.

18. The method according to claim 16 wherein the pharmaceutical composition is administered in the form of a powder, tablet, capsule, dissolving strips, lozenge, syrup, suspension, emulsion, tincture, elixir or effervescent formulation.

19. A method of preventing or improving a neurological deterioration in a subject in need thereof, comprising administering the composition of claim 1 at a dosage of at least 0.5 mg of the long chain fatty acyl tryptamide, and at least 1.5 mg of the caffeine,
wherein the neurological deterioration is selected from decline in memory, mild cognitive impairment, dementia, reduced alertness, slow movements, Parkinsonian signs, tremor, poor coordination of movements, anosmia, REM sleep behavior disorder, or a genetic locus identified as a risk factor for neurodegenerative disease.

20. A method of reducing at least one of α-synuclein aggregation or tau protein aggregation in the central nervous system tissue of a subject in need thereof, comprising administering the composition according to claim 1.

21. A method of reducing at least one of phosphorylated α-synuclein aggregate levels or phosphorylated tau protein aggregation levels, in at least one of the central or peripheral tissues of a subject in need thereof, comprising administering the composition of claim 1.

22. The method according to claim 20, wherein the tissue has a pathology selected from Lewy bodies, Lewy neurites, neurofibrillary tangles, amyloid plaques, or other pathologic protein aggregates or inclusions.

23. A method of reducing the levels of inflammatory markers in a subject in need thereof, comprising administering the composition of claim 1.

24. The method according to claim 23, wherein the inflammatory markers are representative of at least one of microgliosis or astocytosis.

25. A method of increasing the levels of dopamine in a subject in need thereof, comprising administering the composition of claim 1.

26. A method of protecting and preserving the tyrosine hydrolase (TH) positive dopaminergic neurons in a subject in need thereof, comprising administering the composition of claim 1.

27. A method of increasing the levels of methylated protein phosphatase 2A (PP2A) in a subject in need thereof, comprising administering the composition of claim 1.

28. A method of decreasing the levels of demethylated protein phosphatase 2A (PP2A) in a subject in need thereof, comprising administering the composition of claim 1.
